(19) United States
(12) Patent Application Publication
(10) Pub. No.: US 2024/0188884 A1
(43) Pub. Date: Jun. 13, 2024

(54) PRESSURE DYNAMIC DEVICE FOR TREATMENT OF CHEST WALL PAIN AFTER THORACIC SURGERY OR INJURY, AND METHOD OF USE

(71) Applicant: Norman A. Paradis, Putney, VT (US)

(72) Inventor: Norman A. Paradis, Putney, VT (US)

(21) Appl. No.: 18/581,278

(22) Filed: Feb. 19, 2024

Related U.S. Application Data
(63) Continuation-in-part of application No. 17/532,992, filed on Nov. 22, 2021, now Pat. No. 11,903,726.
Application No. 17/532,992 is a continuation of application No. 15/051,383, filed on Feb. 23, 2016, now Pat. No. 11,179,098.
(60) Provisional application No. 62/119,588, filed on Feb. 23, 2015.

(51) Int. Cl. A61B 5/00; A61B 5/08; A61B 5/113; A61H 9/00; A61H 31/00; A61H 31/02

(52) U.S. Cl. CPC A61B 5/4836; A61B 5/08; A61B 5/1135; A61H 9/0078; A61H 2031/002; A61H 31/02; A61H 2201/0103; A61H 2201/1238; A61H 2201/5087; A61H 2230/405; A61H 2230/655

(57) ABSTRACT

A device and method for treating chest wall injuries, including thoracic surgeries, rib fractures, flail chest injuries, or surgical incisions so as to lessen pain experienced by patients. The device and method can include creating a localized airtight compartment external to the chest wall and fully covering the area of injury, varying the pressure within the compartment, and providing dynamic real-time counter forces that act reciprocal to the intrathoracic pressure changes that occur during ventilation. In a preferred embodiment, the apparatus has the capability of sensing the patient's chest wall motion created by ventilation, a pressure control component capable of varying the pressure within the airtight compartment such that it opposes pressure changes within the chest. The pattern of positive and negative pressures may be adjusted based on the patient's subjective sense of their pain.

### RELATED APPLICATIONS

This application is a continuation-in-part of co-pending U.S. patent application Ser. No. 17/532,992, filed Nov. 22, 2021, entitled A SYSTEM FOR DYNAMICALLY STABILIZING THE CHEST WALL AFTER INJURY, FRACTURE, OR OPERATIVE PROCEDURES, which is a continuation application of co-pending U.S. patent application Ser. No. 15/051,383, filed Feb. 23, 2016, entitled A SYSTEM FOR DYNAMICALLY STABILIZING THE CHEST WALL AFTER INJURY, FRACTURE, OR OPERATIVE PROCEDURES, which claims the benefit of co-pending U.S. Provisional Application Ser. No. 62/119,588, entitled A SYSTEM FOR DYNAMICALLY STABILIZING THE CHEST WALL AFTER INJURY, FRACTURE, OR OPERATIVE PROCEDURES, filed Feb. 23, 2015, the entire disclosures of each of which applications are herein incorporated by reference.

### FIELD OF THE INVENTION

The invention disclosed herein relates in general to the fields of devices used in medicine, surgery and emergency trauma therapy, and in particular to methods for alleviating chest wall pain and the associated morbidity after thoracic surgery.

### BACKGROUND OF THE INVENTION

Every year, at least serval hundred thousand patients undergo thoracic surgery worldwide. Many others suffer thoracic injuries. Effective treatment of post-surgical and post injury pain is important to achieving the best possible outcomes and depends largely on proper pain management during the first few days after a thoracic surgery or injury. Postoperative pain after surgery is a significant complication that negatively affects outcomes and increases health care costs.

Similar to postoperative pain, pain associated with injury to the thorax may interfere with healing and impair outcomes. Some of the major types of traumatic thoracic injuries include:
1. Rib fractures: This is one of the most common types of traumatic thoracic injuries. It is usually caused by blunt force trauma to the chest.
2. Sternal fractures: This is a fracture of the breastbone that can be caused by a direct blow to the chest.
3. Pulmonary contusions: This is a bruising of the lung tissue, which can be caused by blunt trauma to the chest.
4. Pneumothorax: This is a condition in which air enters the pleural cavity, causing the lung to collapse.
5. Hemothorax: This is a condition in which blood collects in the pleural cavity, usually as a result of trauma.
6. Flail chest: This is a condition in which a segment of the rib cage becomes detached from the rest of the chest wall, causing paradoxical movement of the chest wall during respiration.
7. Cardiac contusions: This is a bruising of the heart muscle, which can be caused by blunt trauma to the chest.
8. Traumatic aortic injury: This is an injury to the aorta, the largest artery in the body, usually caused by blunt force trauma to the chest.

For the purposes of this discussion, pain caused by any of the above can be referred to as post thoracic surgery and/or injury pain (PTSIP).

During normal ventilation, intrathoracic pressure typically fluctuates with the respiratory cycle. When a person inhales, the diaphragm contracts, and the intercostal muscles expand the chest wall. This results in a negative pressure within the thorax, which helps to draw air into the lungs. As the person exhales, the diaphragm and intercostal muscles relax, causing the chest wall to recoil and the lungs to deflate. This leads to a positive pressure within the thorax, which helps to expel air from the lungs. The magnitude of the negative pressure varies, but it typically ranges from −5 to −10 cm H2O (centimeters of water). This negative pressure helps to draw air into the lungs and also facilitates venous return to the heart.

During expiration, the diaphragm and intercostal muscles relax, causing the chest wall to recoil and the lungs to deflate, resulting in a positive pressure within the thorax. The magnitude of the positive pressure also varies, but it typically ranges from +5 to +10 cm H20. This positive pressure helps to expel air from the lungs and impedes venous return to the heart.

Overall, the pattern of intrathoracic pressure during normal ventilation is a cyclic oscillation between negative and positive pressure, which helps to facilitate the exchange of gases in the lungs and the return of blood to the heart.

It is worth noting that these pressure values are just estimates and can vary depending on several factors, such as the person's lung compliance, airway resistance, and respiratory rate. However, the cyclic oscillation between negative and positive pressures during normal ventilation is critical for proper gas exchange and circulation.

Of importance to the issue of PTSIP, the ventilatory cycle creates intrathoracic negative and positive pressures that act upon thoracic incisions and injuries in a manner that exacerbates pain.

PTSIP can be an acute pain of high intensity, proportional to the type and degree of procedure or injury. The associated inflammatory response triggered within the tissue by surgical trauma, bone fracture and dislocation, dissection, and/or tissue retraction can exacerbate the pain.

The degree of PTSIP is influenced by various factors that increase or decrease the patient's pain threshold, such as the location of the surgery, its extent, the degree of tissue traumatization, the direction of skin incision, preoperative anxiety level, and the analgesic techniques used in the perioperative period. Pain after cardiac surgery is most severe during the first 24 hours and decreases on subsequent days, and that is why it is called “self-limiting”. Risk factors after cardiac surgery include young age, female gender, preexisting pain, anxiety, a catastrophizing mindset, higher body mass index, and a history of osteoarthritis.

Improving patient outcomes after cardiac surgery is to some extent dependent upon the proper management of post-cardiac surgery pain. Managing acute postoperative pain is crucial as failure to manage acute postoperative pain can lead to: prolonged hospitalization, increased use of opiates and their associated toxicity, progression to chronic pain, infections, immunosuppression, and slower wound healing. Despite significant efforts to treat acute pain, moderate to severe intensity acute pain remains common, affecting nearly 75 percent of cardiac surgery patients.

The pain is generally worse on the first day after surgery or injury and subsides during the following days. In addition, the pain continues to decrease in severity over time, with only 16 percent still experiencing it as chronic pain after 12 months and only 9.5 percent after 24 months.

The patient's sense of pain is contemporaneous to each surgical procedure or thoracic injury. The severity and duration of pain are often directly correlated with the extent of the operation or injury because it results from the acute tissue, organ and skeletal injury. In situations of significant trauma, in addition to surface and deep somatic pain, the visceral component of postoperative pain is also implicated. Inflammatory mediators and smooth-muscle contraction, which compress and strain the visceral tissues, both contribute to the development of this discomfort. A variety of variables that raise or lower the nociceptive threshold have a major impact on the patient's level of pain. These factors include the surgery's site, its scope, the degree of tissue traumatization, the orientation of the skin incision, the patient's level of preoperative anxiety, and the perioperative analgesic treatments.

Despite being treatable, pain after thoracic surgery or injury is moderate to severe in up to 75% of patients. In addition to extending hospital stays, pain can also result in considerable morbidity, such as psychological discomfort. Cardiac surgery is associated with significant postoperative discomfort on days (POD) 1 and 2. While discomfort lessens throughout the course of the recovery period, on POD 4 approximately half of patients still report having significant pain when at rest. Throughout the postoperative period, the pain's location shifts. The main surgical site experiences the bulk of severe pain in the early postoperative phase; however, after POD 2, pain in the lower extremities from vein extraction and in the shoulders becomes additional significant regions of pain. Through POD 4 and POD 6, deep breathing, turning, moving, coughing, and employing incentive spirometry are all linked to discomfort. The most common issue after heart surgery remains to be uncontrolled acute postoperative discomfort.

Etiology Of Pain After Surgery: Cardiac surgery patients may have significant postoperative pain with several etiologies. Skin incision, dissection, sternal retraction, internal mammary artery graft preparation, chest drain insertion, endotracheal intubation, and sternal wires all cause direct tissue damage and trigger the production of a variety of pro-inflammatory mediators, such as nitric oxide and cytokines. These mediators generate nociceptive pain by stimulating afferent nociceptive fibres. The inflammation brought on by cardiac bypass and anesthetic medications may make nociceptive discomfort worse.

Tissue degradation, intercostal nerve injury, scarring, rib fractures, sternal infection, stainless steel sutures, and/or costochondral avulsion are all causes of post-sternotomy discomfort, which is a consequence of heart surgery. Furthermore, sternal retraction, particularly while the internal mammary artery is being harvested, may fracture or dislocate ribs and is a major source of musculoskeletal discomfort. Costochondritis may be caused by the misalignment of the ribs connected to the replacement sternum. Intercostal chest drain implantation may cause pleuritic discomfort. Incisional and traumatic injury pain predominates in the early postoperative period, but when that pain passes, musculoskeletal pain takes over. Occasionally, discomfort may linger for a long time after surgery. The most common cause of chronic pain is traumatic or inflammatory nerve damage, which causes neuropathic pain. Sternal wires may also set off an excessive fibrotic reaction that entraps sensory neurons and causes excessive inflammation.

There are several incisions that are commonly used in cardiac and thoracic surgery, depending on the specific procedure being performed. Some of the major incisions used can include:
1. Median sternotomy: This is the most common incision used in cardiac surgery. It involves dividing the sternum (breastbone) vertically down the middle to access the heart.
2. Thoracotomy: This incision involves making a cut through the side of the chest wall to access the lungs or other thoracic organs. There are several types of thoracotomy incisions, including anterior, lateral, and posterior.
3. Minimally invasive incisions: These are smaller incisions that are used in certain types of cardiac and thoracic surgery to minimize trauma to the surrounding tissue. Examples include small incisions made between the ribs (thoracoscopy) or in the groin area (femoral artery access).
4. Transverse thoracotomy: This incision is made horizontally across the chest and is used in certain procedures that require access to the middle or lower portion of the chest.
5. Clamshell incision: This incision involves making a Y-shaped cut in the chest to provide access to both the heart and lungs. It is typically used in emergency situations where rapid access is needed.

The pattern of PTSIP associated with each of these incisions may be different.

Etiology Of Pain After Injury: The causes of pain after thoracic injury can vary depending on the type and severity of the injury. Here are some of the common causes of pain after thoracic injury:
1. Rib fractures: Pain after rib fractures is usually caused by the movement of the broken bones during breathing, coughing, or other physical activities.
2. Pulmonary contusions: Pain after pulmonary contusions is usually caused by inflammation and bruising of the lung tissue, which can cause discomfort and difficulty breathing.
3. Pneumothorax: Pain after a pneumothorax is usually caused by the collapse of the lung, which can cause sharp, stabbing pain in the chest.
4. Hemothorax: Pain after a hemothorax is usually caused by the accumulation of blood in the pleural cavity, which can put pressure on the lung and other organs, causing discomfort and pain.
5. Flail chest: Pain after a flail chest is usually caused by the paradoxical movement of the chest wall during respiration, which can cause severe pain and difficulty breathing.
6. Cardiac contusions: Pain after a cardiac contusion is usually caused by the bruising of the heart muscle, which can cause chest pain, shortness of breath, and other symptoms.
7. Traumatic aortic injury: Pain after a traumatic aortic injury is usually caused by the tearing or rupture of the aorta, which can cause severe chest pain, shortness of breath, and other symptoms.

Deleterious effect of post-cardiac surgery pain: Numerous negative effects on recovery are causally linked to high post-operative pain levels. Patients with pain breathe more quickly and shallowly, since discomfort prevents satisfying coughing and deep breathing. Mucus builds up as a consequence, putting patients at risk for pneumonia and atelectasis, which may lead to extended hospital admissions, mechanical ventilation, and antibiotic use. In addition to being less likely to move about, patients who are in pain also tend to sit up straight, which raises the risk of deep venous thromboembolism owing to prolonged immobilization. This may worsen tissue-induced muscle atrophy and lengthen the time needed to regain mobility sufficient for discharge in addition to encouraging atelectasis.

Postoperative cardiac surgery pain prevents regular coughing and breathing, putting patients at risk for various pulmonary conditions like pneumonia and atelectasis. Pain also activates the autonomic nervous system, and together with pulmonary complications, it creates an unfavorable cardiovascular state, which predisposes the patient to conditions like atrial fibrillations and myocardial infarction.

Postoperative pain often leads to delayed ICU and hospital discharge, increased mechanical ventilation, nosocomial infections and non-compliance with physiotherapy. Extended lengths of stay and readmissions decrease the performance of hospitals and ICUs. Thus, while acute post-cardiac surgery pain is uncomfortable and debilitating for the patient, it is resource intensive and a health burden for hospitals.

Additionally, pain stimulates the hypothalamic-pituitary adrenal axis and activates the sympathetic nervous system (SNS) which quickens the heart rate and rate of contraction and conduction. This adverse cardiovascular condition might increase myocardial oxygen consumption, which predisposes individuals to ischemic events, and induce arrhythmias, including atrial fibrillation (AF). Depending on the cardiac surgical method, the frequency of postoperative AF varies from 29 to 63 percent, and it may need a readmission to cardiac critical care or the high dependency unit for strict monitoring. Patients often experience worry and discomfort due to pain, which affects their ability to sleep, leaves them exhausted, and lowers their mood. After heart surgery, between 21 and 55 percent of patients get chronic pain syndromes; severe or protracted acute pain is a risk factor for this development. Acute pain that is not well controlled may lead to immunological and neurological alterations that eventually lead to chronic pain.

Management of pain after thoracic surgery or injury: It is common for postoperative pain to go untreated. The under-reporting of pain, inter-individual differences in pain thresholds, and problems of over-analgesia, which may impede breathing, impair consciousness, and have nephrotoxic effects, are just a few of the issues that make treating pain effectively difficult. Evidence has also shown the value of multi-modal analgesia in the management of post-operative pain. Accordingly, a combination of analgesics, such as neuropathic agents, opioids, paracetamol, and/or non-steroidal anti-inflammatory medicines (NSAIDs), may speed up recovery, enable early mobilization, and reduce the need for opioids while also having less negative side effects.

The potential benefits of adequate acute analgesia for post-cardiac surgery pain are significant. Effective management of acute postoperative pain helps the patient feel comfortable, improves their sense of well-being, speeds up recovery and promotes early mobilization after surgery. In addition, acute analgesia prevents complications like chronic pain, respiratory infections, and cardiovascular conditions like atrial fibrillation.

In addition, acute analgesia helps with earlier patient mobility and compliance with physical therapy, making the patient's health status satisfactory for earlier discharge and reducing resource utilization in hospitals and ICUs. It also improves patients' mental health and promotes psychological satisfaction after surgery. Furthermore, it prevents patients' dependence on opioids for pain relief.

Analgesics for post-cardiac surgery pain: One of the most important aspects of providing adequate post-surgical patient care is pain control in the postoperative phase. Inadequate analgesia throughout the recovery phase may result in a variety of negative issues, including hemodynamic instability (hypertension, tachycardia, and vasoconstriction), immunologic disruption (impaired immune response), metabolic (extensive catabolism), and hemostatic disorder (platelet activation). It can be challenging to get the best pain treatment after heart surgery. Numerous procedures, such as sternotomy, thoracotomy, leg vein harvesting, pericardiotomy, or chest tube insertion, among others, may cause pain. Inadequate analgesia during the postoperative period might worsen hemodynamic, metabolic, immunologic, and hemostatic changes, which can increase morbidity. Therefore, both after noncardiac surgery and cardiac surgery, intensive postoperative pain management may improve outcomes in high-risk patients.

Pharmacological management of PTSIP involves targeting the various pathways and mechanisms of pain using non-opioid and opioid analgesics.

The most commonly used non-opioids include nonsteroidal anti-inflammatory drugs (NSAIDs), metamizole, and paracetamol. NSAIDs decrease the production of inflammatory cytokines and chemokines such as prostaglandins. This may be particularly helpful in the acute phase of injury for patients, but also plays a role in chronic musculoskeletal pain and associated flares. Opioid analgesic agents are frequently used to manage postoperative pain. Morphine given intravenously, especially if administered through patient-controlled devices, successfully relieves pain during the first postoperative days. Remifentanil has been assessed for its potential association with postoperative hyperalgesia. However, controversial evidence seems to conclude that remifentanil does not exacerbate pain in the hours immediately following cessation. Similar considerations can be made for Sufentanil and Hydromorphone.

Adequate postoperative analgesia reduces postoperative morbidity, eliminates needless patient suffering, shortens hospital stays after surgery, and perhaps lowers costs. There are several ways to achieve postoperative analgesia.
1. Intravenous administration of opioids and non-steroidal anti-inflammatory drugs, alpha-adrenergic drugs.
2. Infiltration of local anesthetics.
3. Nerve blocks.
4. Epidural techniques.
5. Intrathecal techniques

Opioids: Opioid analgesics have historically been used to relieve pain after surgery. These continue to be the medical standard for managing moderate to severe acute pain. Fentanyl, hydromorphone, morphine, oxycodone, oxymorphone, and tramadol are examples of intravenous opioids. Following surgery, oxycodone (OxyContin, Roxicodone, among others) and oxycodone with acetaminophen (Percocet) are a few examples of opioids that may be provided as pills. It is possible to give morphine orally, intramuscularly, subcutaneously, and intravenously. Morphine has a long-lasting analgesic effect, is affordable, and is simple to adjust the dosage of.

Analgesic Medications. Opioids like morphine have long been considered the gold standard and used extensively as these offer potent analgesic effects with rapid onset after intravenous (IV) administration. Opioids' however have significant toxicities and their dosage should be minimized.

While opioids are the initial primary analgesia following cardiac surgery, other medications may also be effective at managing acute pain. The severity of discomfort varies significantly among patients due to different causes and risk factors, and not everybody needs an opioid prescription. Evidence has shown that multimodal analgesia, involving a combination of analgesics including neuropathic agents, opioids, paracetamol and/or NSAIDs, may be safer and more effective in managing acute postoperative pain. Paracetamol and NSAIDs like naproxen and ibuprofen can be effective during the acute phase of pain and may also help with chronic musculoskeletal pain. Anticonvulsant gabapentinoids like pregabalin and gabapentin may be a good option, especially for neuropathic pain; however, the research so far presents some conflicting results, with some studies indicating a potential benefit while others suggest them to be ineffective. In addition, serotonin and norepinephrine reuptake inhibition (SNRIs) like duloxetine and milnacipran and tricyclic antidepressants (TCAs) like nortriptyline and amitriptyline may also help with neuropathic or musculoskeletal pain. Dexamethasone may also be considered, but it may or may not be effective at reducing postoperative median sternotomy pain.

Postoperative local anesthetic infiltration: Thoracotomy or sternotomy incisions during cardiothoracic surgery cause severe pain. A decline in pulmonary function and a rise in cardiac morbidity are linked to these incisions. At the conclusion of the surgery a catheter may be left at the site of the median sternotomy incision for the infusion of local anesthetic medications. This method may improve analgesia and be associated with early ambulation, and shorter hospital stays, but concerns have been expressed about the catheter also being associated with tissue necrosis and infection.

Paravertebral Intercostal Block: These blocks are widely used for post-cardiac surgery analgesia. Intercostal nerve blocks have significantly reduced the need for additional analgesics. In specialized procedures, the PVBs of the thoracic segments are used to relieve both intraoperative and postoperative pain. The spinal nerve comes from the intervertebral foramen adjacent to the vertebral canal in the thoracic area, where the local anesthetic is administered. Local anesthetics move both caudally and rostrally from the injection site. Advantages of these blocks include: Low incidence of complications, quick hospital stay, easy to learn, safe to present on respiratory and sedated patients. PVB are often used unilaterally and are beneficial for individuals who are more vulnerable to neuraxial methods (coagulopathies). Additionally, vascular, thoracic, and bilateral breast procedures employ bilateral PVBs.

Intrapleural block: Intrapleural analgesia is a type of pain management technique that involves the injection of local anesthetic medication into the pleural cavity, which is the space between the lungs and the chest wall. Local anesthetic medication may be delivered through bolus or continuous infusion via an intrapleural catheter inserted between the visceral and parietal pleura. This method frequently results in systemic absorption of local anesthetic drug resulting in toxicity. According to a clinical study involving patients who underwent thoracic surgery through a thoracotomy incision, 0.25 to 0.5 percent bupivacaine may help patients feel less pain after thoracic surgery, and a 0.25 percent bupivacaine intrapleural block is perfect, secure, and offers the right amount of postoperative analgesia.

Intrathecal analgesia: In patients having cardiothoracic surgery, physicians employed intrathecal analgesia for the first time in 1980. The benefits of using this method for analgesia have been well-explained. The patients are substantially more compliant, relaxed, and able to retain their movement in bed more readily.

Intrathecal analgesia has been utilized in the majority of clinical investigations to provide postoperative patients with extended analgesia. Injecting intrathecal sufentanil, fentanyl, and local anesthetics has been recommended in several clinical trials for intraoperative anesthesia and analgesia. Uncertainty exists over the ideal intrathecal morphine dosage that will produce the best possible surgical analgesia with the fewest adverse medication reactions. The pain relief produced by higher intrathecal morphine dosages is more intense and lasts longer, but it may also result in more frequent unfavorable medication side effects. Although this method is meant for analgesia and sympatholysis, it may also result in side effects such as bradycardia, hypotension, respiratory depression, nausea, vomiting, and itching.

Epidural analgesia: Epidural analgesia is associated with greater analgesia, faster extubation time, better hemodynamics, less respiratory problems, and superior left ventricular function in cardiothoracic surgery. Hypotension, epidural abscess, epidural hematoma, and epidural abscess are significant side effects of this procedure. The low prevalence of these consequences is encouraging for pregnant women and advantageous for severe discomfort.

Wound Infiltration: Recently, multimodal analgesia schemes for postoperative pain control following various surgical procedures under regional anesthesia have once again included single wound infiltration or continuous wound infiltration through catheters placed into the surgical wound. Although complications from wound infiltration are uncommon, they may occur and include bruising, hematoma, wound infection, and local anesthetic toxicity. Concerns related to infection is likely one of the biggest barriers to wound infiltration. However published data reveal low infection risk in both active (0.7 percent) and control groups (1.2 percent).

During wound infiltration, accidental superficial vascular puncture might result in superficial bruising or hematoma. Although bruises heal naturally, patients should be made aware of this risk. CWI extends the length of the procedure and necessitates the insertion of specific catheters, increasing the expense and posing a risk of infection. Wound infection, catheter leaking, kinking or blockage, failure to infuse owing to occlusion, inadvertent removal, and improper tube management are among the reported CWI problems.

The need to create negative intrathoracic pressures so as to allow ventilation has necessitated that the human chest be a rigid structure. The is achieved via the ribs, the spine and connecting cartilage, all of which form components of the thorax. The rigid and semi-rigid components of the thorax may be damaged as a result of injury or operative surgical procedure. Rib fractures may involve one or more than one ribs. Surgical entry into the chest may cause fracture or injury to the ribs or sternum.

During ventilation, intrathoracic pressure alternates between positive and negative pressure so as to achieve air movement. After injury to the chest wall, this phasic change in intrathoracic pressure is associated with pain. This pain may cause splinting and interference with normal ventilation.

Flail chest injury in particular is among the most serious traumatic injuries of the thorax. In flail chest injury a sufficient number of ribs are broken in multiple locations such that a portion of the chest wall is no longer structurally attached to the thorax. It is among the most clinically challenging of chest wall injuries not involving actual penetration and may be associated with severe pain. Flail chest injury is significant in particular because pathologic movement of the free segment may result in additional injury. The ends of the fractured ribs may lacerate intercostal vessels and lung tissue. Injury to the underlying lung may cause and exacerbate pulmonary contusion. Movement of the free segment is very painful to the patient. Movement of the free segment may be paradoxical to the remainder of the chest wall with ventilation. When the patient inhales and exhales, the uninjured chest wall moves outward and inward respectively. The free segment in flail chest injury may move reciprocally, moving in with inspiration and out with expiration. This paradoxical movement may be particularly painful and will act to exacerbate visceral injuries. Currently, Flail chest injury is definitively stabilized surgically in the operating room. There is currently no effective noninvasive method or device to treat, or even stabilize, flail chest injury.

Rib fractures are among the most common chest wall injuries and are associated with significant morbidity and pain. Cardiac surgery is frequently undertaken via an artificial cut through the sternum called a median sternotomy. Both rib fractures and median sternotomy are painful for patients and this pain is difficult to treat. They are also associated with significant morbidity as the pain associated with each of the components of the ventilatory cycle cause the patient to limit chest wall motion which interferes with pulmonary toilet. Similarly, this pain interferes with the normal sighing required for pulmonary toilet.

Pain Is Multifactorial: Physical pain is a complex experience that is subjective and multifactorial. This means that pain is not just a result of a physical injury or damage to the body, but it is also influenced by a variety of psychological and social factors. Biological factors include things such as genetics, injury severity, and medical conditions that affect pain. Psychological factors include emotions, anxiety, depression, and attentional focus. Social factors include cultural background, social support, and the expectations of others.

Subjectivity of pain refers to the fact that pain is a personal experience that varies from person to person. Even though two people may have the same injury, they may experience pain differently. Factors such as age, gender, personality, previous experiences with pain, and cultural background can all affect how a person perceives and reports pain.

The pain experienced by wounds can be influenced by various local tissue and skeletal factors. Here are some examples:
1. Size and location of the wound: The size and location of the wound can affect the pain experienced. Larger wounds or those located in areas with a higher concentration of nerve endings (such as the fingers, toes, and face) may be more painful.
2. Depth of the wound: Deeper wounds may be more painful because they involve a greater amount of tissue damage and may affect underlying structures, such as nerves or bone.
3. Inflammation: Inflammation is a normal response to injury, but it can cause pain by activating nerve endings and increasing pressure within the wound.
4. Infection: Infections can cause pain by triggering the immune system response and leading to inflammation, and by damaging tissue and causing nerve irritation.
5. Type of tissue damage: Different types of tissue damage can cause different types of pain. For example, a laceration or cut may cause sharp, stabbing pain, while a burn may cause a more intense, burning pain.
6. Bone involvement: If a wound involves damage to bones or joints, it can cause significant pain due to the large number of nerve endings in these areas.
7. Presence of foreign objects: If there are foreign objects (such as dirt or debris) in the wound, they can cause irritation and pain.
8. Movement and pressure: Movement or pressure on the wound can exacerbate pain. For example, a wound on the foot may be more painful when walking or standing.
9. Nerve damage: Damage to nerves can cause persistent pain, even after the wound has healed.
10. Tissue ischemia: Reduced blood flow to the wound can cause tissue ischemia, leading to tissue damage and increased pain

Overall, many local tissue and skeletal factors can exacerbate the pain experienced by wounds. With respect to this discussion, the exacerbation of wound pain by movement and pressure is relevant.

The changes in intrathoracic pressure created during inspiration exacerbate the pain of rib fractures, median sternotomy, and flail chest injuries. In the case of flail chest injury the changes in intrathoracic pressure associated with the ventilatory cycle exacerbate movement of the free segment and contribute to further injury.

Measurement of Ventilation: It is now possible to measure ventilation noninvasively using techniques such as impedance, and plethysmography, among others. Here are some of the current techniques.
1. Capnography: Capnography is a non-invasive technique used to measure the amount of carbon dioxide (CO2) in exhaled breath. This technique is used to monitor ventilation and can provide information on the adequacy of ventilation and/or the effectiveness of Non-Invasive Ventilation (NIV).
2. Transcutaneous carbon dioxide monitoring (TcPCO2): TcPCO2 is a non-invasive method that measures the partial pressure of CO2 in the blood through the skin. This technique can be used to monitor ventilation and to adjust NIV settings.
3. Pulse oximetry: Pulse oximetry is a non-invasive technique used to measure the oxygen saturation of the blood. This technique can provide information on the adequacy of oxygenation and can help to adjust NIV settings.
4. Respiratory inductance plethysmography (RIP): RIP is a non-invasive technique that measures chest wall movement and can be used to estimate lung volume and breathing effort. This technique can provide information on the effectiveness of NIV and can be used to adjust settings.
5. Transcutaneous oxygen monitoring (TcPO2): TcPO2 is a non-invasive method that measures the partial pressure of oxygen in the blood through the skin. This technique can provide information on the adequacy of oxygenation and can help to adjust NIV settings.
6. Impedance pneumography: Impedance pneumography is a non-invasive technique that measures changes in the electrical impedance of the chest wall during breathing. This technique can provide information on breathing patterns and can help to adjust NIV settings.
7. Magnetic resonance imaging (MRI): MRI is a non-invasive imaging technique that can provide information on lung volume, regional ventilation, and breathing patterns. This technique is not commonly used for routine NIV monitoring but may be used in research settings.

### Limitations of Current Techniques

Despite the progress made in medicine in the field of pain pathophysiology and its treatment, patients still experience PTSIP. Poor pain treatment may lead to negative pulmonary, cardiac, gastrointestinal, musculoskeletal, endocrine, and psychological effects in the form of atelectasis, pneumonia, tachycardia and increased O2 consumption, muscle weakness and disuse, hyperglycemia, and depression. Poor pain treatment may lead to chronic intractable pain.

Adequate pain control is complex, a challenge that stems from a combination of poor reporting of pain, inter-individual variation in pain thresholds and the side-effects of strong, particularly opioid, analgesics, which can reduce consciousness, impair breathing and cause nephrotoxic effects.

Heart Pillow: While the heart pillow can provide some comfort and support after surgery or injury to the sternum, there is limited scientific evidence to support its use. While some studies have suggested that the use of a heart pillow may help reduce pain and discomfort after median sternotomy surgery by providing support to the chest wall and reducing strain on the incision site, there is significant risk that this is principally placebo and Hawthorne study effect as other studies have not found significant benefits associated with the use of a heart pillow, and it is not currently considered a standard part of postoperative care.

The sternum support harness has limited application because it may interfere with ventilation. Patients who have difficulty breathing or have other medical conditions that affect their ability to wear a chest strap or harness are a not good candidates for this device.

Opioids are often the first analgesics used after heart surgery, and they are particularly helpful for pain that occurs when resting. They do, however, have a constrained therapeutic range and various undesirable side effects, including nausea, vomiting, pruritus, constipation, and urinary retention, when used at higher quantities. Large opioid doses may also worsen respiratory issues, limit mobility because of increased tiredness, and encourage addiction. Opioids may exacerbate renal failure and result in post-operative ileus. Because of this, despite their considerable power, they should only be used sparingly and when necessary.

NSAIDs may also be used after surgery, though, they are associated with an increased risk of post-operative bleeding, and precipitate acute kidney injury, particularly in patients undergoing cardiopulmonary bypass (CPB).

Paravertebral nerve block is associated with a number of complications including:
1. Nerve damage: If the needle used for the block penetrates or damages the spinal nerves, it can cause nerve damage and other serious complications.
2. Pneumothorax: If the needle penetrates the lung tissue, it can cause air to leak into the pleural cavity, resulting in a collapsed lung (pneumothorax).
3. Hematoma: In rare cases, the injection site may bleed excessively, causing a collection of blood (hematoma) to form in the area around the injection site.
4. Infection: Anytime a needle is inserted into the body, there is a risk of infection. If an infection develops, it can cause pain, swelling, and other symptoms.
5. Allergic reaction: Some people may be allergic to the local anesthetic medication used for the block, which can cause an allergic reaction.
6. Respiratory depression: In rare cases, the medication used for the block may cause respiratory depression, which can result in breathing difficulties and other serious complications.

Intrathecal anesthesia, also known as spinal anesthesia, is associated with some potential complications including:
1. Headache: One of the most common complications of intrathecal anesthesia is a headache caused by a leak of cerebrospinal fluid through the puncture site in the spinal cord.
2. Nerve damage: If the needle used for the injection penetrates or damages the spinal nerves, it can cause nerve damage and other serious complications.
3. Infection: Anytime a needle is inserted into the body, there is a risk of infection. If an infection develops, it can cause pain, swelling, and other symptoms.
4. Allergic reaction: Some people may be allergic to the medication used for the intrathecal injection, which can cause an allergic reaction.
5. Respiratory depression: In rare cases, the medication used for the injection may cause respiratory depression, which can result in breathing difficulties and other serious complications.
6. Blood pressure changes: Intrathecal anesthesia can cause changes in blood pressure, which can be dangerous for people with certain medical conditions.
7. Back pain: Some people may experience back pain or discomfort at the injection site after the procedure

Epidural analgesia is associated with a number of potential complications including:
1. Nerve damage: If the needle used for the injection penetrates or damages the spinal nerves, it can cause nerve damage and other serious complications.
2. Infection: Anytime a needle is inserted into the body, there is a risk of infection. If an infection develops, it can cause pain, swelling, and other symptoms.
3. Allergic reaction: Some people may be allergic to the medication used for the epidural injection, which can cause an allergic reaction.
4. Blood pressure changes: Epidural analgesia can cause changes in blood pressure, which can be dangerous for people with certain medical conditions.
5. Headache: In rare cases, the injection can cause a leak of cerebrospinal fluid through the puncture site in the spinal cord, resulting in a headache.
6. Back pain: Some people may experience back pain or discomfort at the injection site after the procedure.
7. Difficulty urinating: Epidural analgesia can sometimes cause difficulty urinating, which may require the use of a catheter.
8. Itching and nausea: Some people may experience itching and nausea as side effects of the medication used for the epidural injection.
9. Urinary retention and itching are frequent adverse effects of thoracic epidural analgesia when opioids are used. A postdural puncture headache may occur after the procedure, depending on the level of expertise of the practitioner. According to reports, paresthesia and neurological issues happen between 0.01 and 0.001 percent of the time. Lower thoracic regions have more issues with catheters and punctures than mid- and higher thoracic parts do.

Intrapleural analgesia is associated with potential complications including:
1. Pneumothorax: If the needle used for the injection penetrates the lung tissue, it can cause air to leak into the pleural cavity, resulting in a collapsed lung (pneumothorax).
2. Bleeding: In rare cases, the injection site may bleed excessively, causing a collection of blood (hematoma) to form in the area around the injection site.
3. Infection: Anytime a needle is inserted into the body, there is a risk of infection. If an infection develops, it can cause pain, swelling, and other symptoms.
4. Allergic reaction: Some people may be allergic to the local anesthetic medication used for the injection, which can cause an allergic reaction.
5. Respiratory depression: In rare cases, the medication used for the injection may cause respiratory depression, which can result in breathing difficulties and other serious complications.
6. Nerve damage: If the needle used for the injection penetrates or damages the intercostal nerves, it can cause nerve damage and other serious complications.

None of the current approaches to PTSIP, either alone or in combination, is nearly effective to prevent the morbidity associated with the condition. It would be desirable to have a device for use after thoracic surgery or injury that, alone or in combination, significantly decreases the pain of PTSIP without additional toxicity or injury.

### PRIOR ART

There is no prior art teaching the local dynamic stabilization of the chest wall by varying of the extra thoracic air pressure. Previous devices and methods are limited to direct mechanical stabilization such as is achieved with screw, clamps or direct pressure. The prior art additionally fails to teach localized treatment of chest wall injuries by creation of an airtight extrathoracic compartment and application of dynamic positive and negative pneumatic counter forces. Additionally, the prior art fails to teach real-time sensing of ventilation as an input to the device controller.

Shaffer (U.S. Pat. No. 8,034,011 B2) teaches dynamic stabilization of only the anterior chest by mechanical pressure applied to the lateral surfaces. He does not teach: localized treatment, creation of an airtight extrathoracic compartment, or application of dynamic positive and negative pneumatic counterforce. Of particular significance, Schaffer teaches away from treatment of conditions other than injury to the anterior chest.

Kochamba (WO1999047085 A1) teaches fixation of tissue and broken bones via a pneumatic bladder. He does not teach: creation of an airtight extrathoracic compartment, sensing of ventilation, or application of dynamic positive and negative pneumatic counterforce.

Others (CN 103431934 A) teach an adjustable air column for fixation or protection of thoracic injury. They do not teach: sensing of ventilation, or application of positive and negative dynamic pneumatic counterforce.

Palmer extensively teaches (U.S. Ser. No. 00/605,9742A, WO 201-515-7154 A1) the use of negative distending force to the anterior chest for purposes such as ventilation. Attachment is adhesive although movement of the device may be pneumatic. He does not teach: creation of an airtight extrathoracic compartment, sensing of ventilation, or application of dynamic positive and negative pneumatic counterforce.

The artificial lungs used during the early to mid 20th century—so called iron lungs—achieved ventilation by varying the extrathoracic pressure of the whole thorax. They did not apply local forces. These were not intended toward the purposes described herein and would be ineffective at treatment of localized injury.

### SUMMARY

This invention overcomes disadvantages of the prior art by providing a novel system and method for minimizing the pain of PTSIP experienced by a patient. The system and method described herein can modulate the pressure within an airtight compartment adjacent to a thoracic injury or surgery site, so that the pressure outside of the chest wall at the incision or injury site counteracts the fluctuating pressure inside of the thorax and acts to mitigate the patient's pain. Changing the pressure outside of the thorax in real time to match the fluctuating pressure inside of the thorax can eliminate or minimize the pressure difference across the thorax and thereby minimize the pain of PTSIP experienced by the patient. The system and method described herein includes sensing patient ventilation, and modulating the pressure in the compartment in response to patient ventilation. The pressure within the airtight compartment can be modulated in real time to counteract the changing pressure within the thorax as the patient breathes in and out. It may additionally have a manual feedback interface for the patient or healthcare provider to adjust the pattern of pressure change within the compartment.

In an embodiment, a pressure modulator for treating pain associated with thoracic incisions and injuries can include a platform adapted for creating a localized airtight compartment external to a chest and fully covering an area of injury, a pump configured to create relative positive pressure and vacuum within the airtight compartment, and a controller adapted to cycle intracavity pressure within the localized airtight compartment so that the enclosed space is under relative vacuum pressure relative to ambient air during patient inhalation and relative positive pressure relative to ambient air during patient exhalation.

The pressure modulator can include a sensor adapted to sense ventilation in real time, wherein the pump dynamically varies the pressure within the localized airtight compartment in response to a sensed ventilation. The pressure modulator can include an adherent material on a patient-side surface of the frame, the adherent material adapted to adhere to the chest and form an airtight compartment between the frame and the chest. The pressure modulator can include an adjustable bladder on a patient-side surface of the frame at a circumference of the frame such that the airtight compartment is adapted to adjust to chest wall anatomy. The pressure modulator can include an intracavity pressure sensor, wherein the controller is adapted to incorporate sensed data from the intracavity pressure sensor to dynamically adjust the intracavity pressure to match changing intrathoracic pressure as the patient is breathing. The pressure modulator can include a manual feedback interface adapted to allow an operator to adjust the pressure within the localized airtight compartment based on a patient's subjective sense of pain. The controller can be adapted to integrate inputs from a manual feedback interface and inputs from one or more sensors to control variation of pressure within the airtight compartment, wherein the sensors are adapted to sense ventilation or pressure within the airtight compartment.

In an embodiment, a pressure modulator for treating chest wall injuries can include a frame adapted for creating a localized airtight compartment external to a chest of a patient and fully covering an area of injury, the frame having a pliable circumferential component with an adhesive, a sensor, a controller adapted to control an air pump, and the air pump, wherein the controller is capable of dynamically varying the pressure within the compartment in real-time in response to data from the sensor so as to provide a dynamic counterforce to the changes in intrathoracic pressure that occur during each ventilatory cycle.

The sensor can be a sensor for detecting ventilation or chest wall motion and, the air pump capable of dynamic variation of the pressure within the localized airtight compartment varies the pressure within the airtight compartment to oppose pressure changes within the chest. The sensor can be a sensor for detecting chest movement, and the air pump capable of dynamic variation of the pressure within the localized airtight compartment varies the pressure within the airtight compartment in response to such motion. The pressure modulator can include an adjustable bladder component on the patient-side surface of the apparatus at its circumference such that the airtight compartment may adjust to chest wall anatomy. The pressure modulator can include an adjustable bladder component on the patient-side surface of the apparatus at its circumference such that the airtight compartment may adjust to chest wall anatomy. The pressure modulator can include an input receiver capable of receiving input signals from other devices measuring ventilation or chest wall movement. The frame can be adjustable to conform to the shape of the chest wall. The pressure modulator can include a patient side sensor capable of detecting or measuring chest wall motion and incorporating that data in the variation of pressure within the airtight compartment. The pressure modulator can include a manual feedback interface for adjusting the variation of pressure within the airtight compartment by an operator based on the patient's subjective sense of pain.

In an embodiment, a pressure modulator for treating chest wall injuries can include a localized airtight compartment adapted to be placed external to a chest and fully covering an area of injury, and a pump adapted to vary a pressure within the localized airtight compartment in real-time so as to provide a dynamic counterforce to changes in intrathoracic pressure that occur during each ventilatory cycle, the dynamic counterforce minimizing a patient's sense of pain by providing a pressure in the localized airtight compartment that opposes movement of the chest wall injury caused by patient breathing.

The pressure modulator can include a sensor adapted to sense in real time ventilation or chest wall motion, wherein the pump is adapted to dynamically vary the pressure within the localized airtight compartment in response to sensed ventilation or chest wall motion data in such a manner that the pressure within the airtight compartment opposes pressure changes within the chest. The pressure modulator can include a sensor adapted to sense in real time patient ventilation, wherein the pump is adapted to dynamically vary the pressure within the localized airtight compartment in response to sensed patient ventilation data in such a manner that the pressure within the airtight compartment opposes intrathoracic pressure. The pressure modulator can include a manual feedback interface adapted to be adjusted by a user based on a patient's subjective sense of pain, wherein the pump is adapted to dynamically vary the pressure within the localized airtight compartment in response to data from the manual feedback interface in such a manner that the pressure within the airtight compartment opposes intrathoracic pressure.

### BRIEF DESCRIPTION OF THE DRAWINGS

For a fuller understanding of the nature of the present invention, reference should be had to the following detailed description and preferred embodiment taken in connection with the accompanying drawing in which:

FIG. 1 is a diagrammatic representation of a pressure modulator, showing a stylized cross section of the pressure modulator placed over a human rib fractured in two locations, according to an illustrative embodiment;

FIG. 2 is a perspective view of a patient laying in a bed with the pressure modulator external to the thorax and applied to minimize pain associated with a median sternotomy incision, according to an illustrative embodiment;

FIG. 3 is a perspective view of a patient laying in a bed with the pressure modulator external to the thorax and applied to minimize pain associated with fractures of the ribs laterally or a lateral thoracotomy incision, according to an illustrative embodiment;

FIG. 4 is chart showing the changes in pressure over time within the thorax and the changes in pressure over time in the cavity of the pressure modulator on the exterior of the thorax, and the relationships between the changing pressures, according to an illustrative embodiment;

FIG. 5 is a schematic representation of various components of a pressure modulator, according to an illustrative embodiment; and

FIG. 6 is a diagrammatic representation of a pressure modulator with a remote base, according to an illustrative embodiment.

### DETAILED DESCRIPTION

Previous therapies for PTSIP have generally been limited to pharmacotherapy, nerve block, or static bracing of the chest wall. As described, even in combination, they are overall ineffective and often associated with significant toxicities.

An extra-thoracic pressure modulator can be used for dynamically treating and stabilizing the chest wall after injury or surgery. The pressure modulator can provide localized treatment of chest wall injuries by creation of an airtight extrathoracic compartment and application of dynamic positive and negative pneumatic counter forces. The pressure modulator can form a closed compartment with the exterior of the chest wall as one wall of the compartment. The space within the closed compartment formed by the chest wall and pressure modulator can be referred to as a cavity, and the pressure modulator can adjust the air pressure or vacuum within that enclosed cavity. Modulating the pressure within the enclosed cavity changes the pressure exerted on the portion of the chest wall that is forming the closed compartment.

As used herein, the word injury can refer to the site of a thoracic surgery, a broken rib, including a flail chest injury, or any thoracic injury that results in pain experienced by the patient when the pressure is different between the interior of the thorax and the exterior of the thorax. The pressure modulator can be positioned over an injury, and the pressure modulator can adjust the air pressure within the enclosed cavity over the injury in a manner reciprocal to the ventilatory respirophasic changes in intrathoracic pressure.

As the patient is breathing in, the patient's diaphragm causes the interior of the thorax to be under a relative vacuum compared to the ambient air outside of the thorax, and this vacuum causes air to be pulled into the lungs from the higher-pressure ambient air outside of the thorax. As the patient is exhaling, the patient's diaphragm increases the pressure inside of the thorax to be greater than the air pressure of the ambient air outside of the thorax, thereby forcing air out of the lungs towards the lower pressure ambient air. However, in the case of injury, this cyclic difference in pressure between the inside of the thorax and the outside of the thorax can cause serious pain. As the inside of the thorax is under a relative vacuum, the chest wall experiences forces that pull inward on the chest wall. Similarly, as the inside of the thorax is under higher pressure than the outside of the thorax, the chest wall experiences forces that push outward on the chest wall. In the event of an injury to the chest wall, this constant pushing out and pulling in on the chest wall can be extremely painful at the injury site of the chest wall.

To reduce the pain felt by the patient, the pressure modulator described herein can be used to reduce the difference in pressure between the inside of the thorax and the area outside of the thorax at the injury or surgery site. In various embodiments, the pressure can be adjusted as a function of the ventilatory cycle. In various embodiments, the pressure can be adjusted as a function of the patient's subjective sense of pain associated with the ventilatory cycle. In various embodiments, the pressure can be adjusted as a function of the ventilatory cycle and as a function of the patient's subjective sense of pain associated with the ventilatory cycle.

Specifically, the pressure modulator can place the cavity of the pressure modulator under relative low pressure, relative to the ambient air outside of the thorax, during patient inhalation, and relative positive pressure, relative to the ambient air outside of the thorax, during patient exhalation. This changing extrathoracic pressure within the closed compartment can counteract the changing intrathoracic pressure to eliminate or decrease the forces that exacerbate pain or cause movement of fractures or free segments. The changing extrathoracic pressure within the closed compartment can counteract the changing intrathoracic pressure by matching the extrathoracic pressure within the cavity to the intrathoracic pressure in real-time as the patient is breathing.

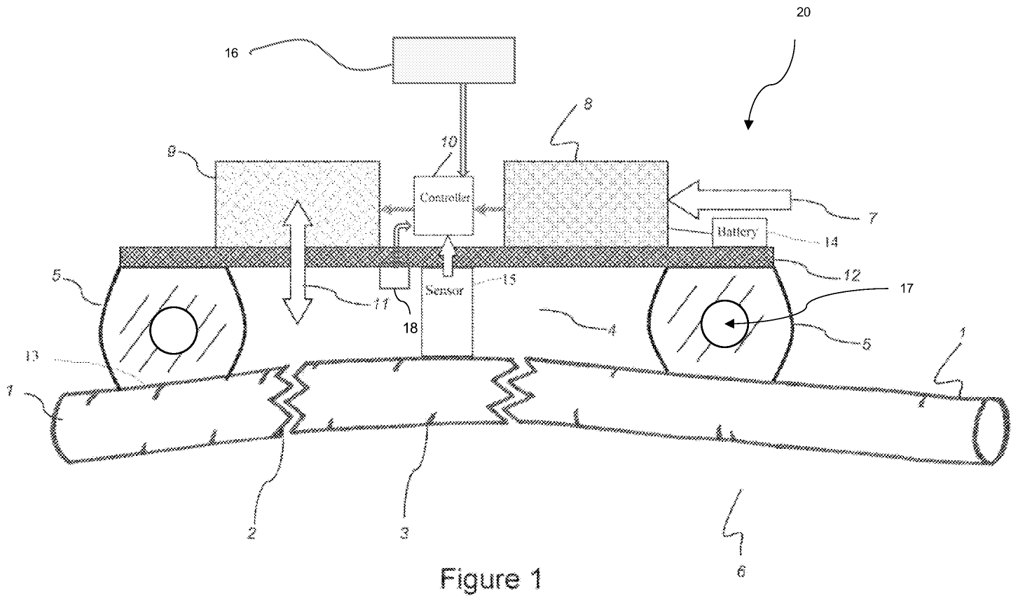

FIG. 1 is a diagrammatic representation of a pressure modulator, showing a stylized cross section of the pressure modulator placed over a human rib fractured in two locations, according to an illustrative embodiment. As shown in FIG. 1, the pressure modulator can be applied to minimize the pain associated with rib fractures, however, it should be clear that the device can be used to minimize the pain associated with a variety of different injuries or medical procedures.

A pressure modulator 20 is adapted to be secured to the patient's thorax 6 to form a closed compartment around a cavity 4. The pressure modulator 20 can be placed over an area of injury, including an incision. By way of non-limiting illustration, as shown in FIG. 1, the pressure modulator can be placed over a broken area of the patient's rib 1, including two break areas 2 and even a detached flail segment 3, however, the pressure modulator can be used for any number of different injuries, including incisions.

A rim wall 5 of the pressure modulator 20 can form a portion of the closed compartment around the cavity 4. The rim wall 5 can be adjustable to expand the size of the pressure modulator to cover larger or smaller areas of injury. At an operator's discretion, the size and shape of the rim wall may be adjusted. In various embodiments, rim wall 5 can be a pliable material and/or an inflatable bladder that can allow the rim wall 5 to adjust to the contours of the exterior of the torso. In various embodiments, rim wall 5 can have a pliable lip and/or adhesive 13 at the lip of the rim wall, where the rim wall 5 meets the torso. The pliable lip and/or adhesive 13 at the lip of the rim wall can help the rim wall to meet the contours of the torso and stick securely to the outside of the torso, thereby helping the pressure modulator to form the airtight closed compartment with the chest wall. In various embodiments, the adhesive can include a hydrogel or similar material that can help to make an airtight seal between the pressure modulator and the torso.

The rim wall 5 can help to form a cavity 4 between the chest wall and a platform 12 of the pressure modulator 20. The pressure modulator can form an irregular shaped closed compartment with a platform 12 held above the chest wall and forming the closed compartment. Although the pressure modulator is described herein as having the rim wall and platform as separate components for ease of description, it should be clear that the rim wall and platform may be a single component that meets the chest wall to form the closed compartment over the area of injury.

An air pump 9 can be mounted on or within the platform 12, and can be used to vary the pressure 11 within the cavity by pumping air back and forth into and out of the cavity. A controller 10 can control the air pump and can direct the air pump 9 to pump air into and out of the cavity to modulate the pressure within the cavity. In various embodiments, components such as pumps, controllers, and/or others can be housed in a remote base that can be separate from the frame, and the components in the remote base can be operatively connected to the platform.

A sensor 8 can measure patient ventilation, and can provide real-time information about the patient ventilation cycle to the controller. In various embodiments, sensor 8 can be one or more sensors. In various embodiments, sensor 8 can receive inputs 7 from additional sensors that can be external to the pressure modulator. By way of non-limiting examples, in various embodiments, the sensor 8 can detect when the patient is inhaling and when the patient is exhaling. In various embodiments, the sensor 8 can detect the depth of patient inhale and exhale. In various embodiments, the sensor 8 can detect the speed of patent inhale and exhale. In various embodiments, the sensor 8 can detect the motion of patient breathing and/or the passage of air into and out of the patient. In various embodiments, the sensor 8 can detect the motion of the patient chest wall associated with breathing. In various embodiments, the sensor 8 can detect various indicators of chest wall motion such as transthoracic impedance.

The controller can receive real-time sensing of the patient's ventilation as an input to the controller. The controller 10 can receive information from sensor 8 about the patient breathing, and the controller 10 can direct the pump 9 to modulate the pressure in the cavity 4 in response to the patent breathing. The pressure modulator can modulate the pressure within the cavity so that it remains approximately the same as the pressure within the thorax throughout the breathing cycle. Throughout the breathing cycle, the pressure within the thorax fluctuates up and down between relative high pressure and relative low pressure compared to the ambient air outside the thorax, and the pressure modulator 20 can keep the pressure within cavity 4 approximately the same as the pressure within the thorax throughout the breathing cycle. As the sensor 8 detects that the patient is breathing in, the controller can determine that a relative low pressure is being created within the thorax, and, the controller can direct the pump 9 to modulate the pressure within the cavity to match the relative low pressure within the thorax. As the sensor 8 detects that the patient is breathing out, the controller 10 can determine that a relative high pressure is being created within the thorax, and the controller can direct the pump to modulate the pressure within the cavity to match the relative high pressure within the thorax.

Information from the sensor 8 such as the speed and/or depth of the patient's breath can be incorporated as the controller controls the pressure within the cavity. By way of non-limiting example, higher speeds of exhalation will result in higher pressure within the thorax. The controller can receive information from the sensor such as a higher-than-normal speed of exhalation in a particular breath, and the controller can incorporate that information to increase the pressure within the cavity to match the higher-than-normal pressure within the thorax. The pressure modulator 20 can monitor patient breathing in real time, and can modulate the pressure within the cavity to match the pressure within the thorax in real time, including for each individual breath as the breath is happening.

Because the pressure modulator cycles the pressure within the closed compartment so as to counteract the effects of ventilation on PTSIP, it may incorporate one or more measurements of ventilation measured by sensor 8, including: capnography, pulse oximetry, respiratory inductance plethysmography (RIP), and impedance pneumography. These biomarker signals may be combined and transformed by the controller 10 into a control signal to the pump 9.

In various embodiments, the pressure modulator can include an intra-cavity pressure sensor 18 that can measure the pressure within the cavity 4. The controller can receive inputs from the intra-cavity pressure sensor 18 that allow the controller to monitor the fluctuating pressure within the cavity 4. The controller can use the intracavity pressure information to help match the intracavity pressure to the intrathoracic pressure in real time as the patient is breathing.

In various embodiments, a paradoxical chest wall motion detector 15 can detect paradoxical motion of a flail segment 3 relative to the rest of the chest wall and the paradoxical chest wall motion detector 15 can provide that information to the controller 10. When a patient breathes in, an intact chest wall can expand outward, and when the patient breathes out, an intact chest wall can move inward. In the case of flail chest injuries, a flail segment 3 can move in the opposite direction of the chest wall. When the patient breathes in and the chest wall is moving out, the relative vacuum or reduced pressure within the torso can pull the flail segment inward relative to the chest wall as the chest moves outward. Similarly, when the patient breathes out and the chest wall moves inward, the relative higher pressure within the torso can push the flail segment outward relative to the chest wall as the chest wall moves inward. As described herein, the pressure modulator 20 can modulate the pressure within the cavity to approximately match the pressure within the thorax to prevent movement of the flail segment. In various embodiments, the controller of the pressure modulator can receive information from a paradoxical chest wall motion detector 15 in real time, and the pressure modulator can use the information from the paradoxical chest wall motion detector 15 to modulate the pressure within the cavity in real time to prevent movement of the flail segment.

By way of non-limiting example, if the paradoxical chest wall motion detector 15 provides information to the controller that the flail segment is moving outward relative to the chest wall, the pressure modulator can increase the pressure within the cavity to prevent the flail segment from moving outward. If the paradoxical chest wall motion detector 15 provides information to the controller that the flail segment is moving inward relative to the chest wall, the pressure modulator can decrease the pressure within the cavity to prevent the flail segment from moving inward. A paradoxical chest wall motion detector 15 can provide information that can be used by the controller to modulate the pressure in the cavity to approximately match the pressure within the thorax in real time as the patient is breathing.

Pain is multifactorial and subjective. It is possible, that the PTSIP experienced by the patient is not fully relieved by simply nulling out chest wall forces by application of perfectly reciprocal external pressures. For this reason, the device may incorporate a control mechanism such as a manual feedback interface 16. In various embodiments, a pressure modulator 20 can include a manual feedback interface 16 that can provide information to the controller 10 from the user. The manual feedback interface 16 can allow a user to adjust the pattern of positive and negative pressure.

The manual feedback interface 16 can allow a patient and/or care provider or other user to adjust the pressure modulation provided by the pressure modulator 20. By way of non-limiting example, the if a patient subjectively feels that the pressure modulator is not providing a high enough pressure within the cavity during the exhalation phase to match the pressure within the thorax, the user may adjust the manual feedback interface 16 to provide information to the controller to increase the pressure within the cavity during the exhalation phase. By way of non-limiting example, the if a patient subjectively feels that the pressure modulator is not providing a low enough pressure within the cavity during the inhalation phase to match the pressure within the thorax, the user may adjust the manual feedback interface 16 to provide information to the controller to decrease the pressure within the cavity during the inhalation phase.

In various embodiments, the user can provide information to the controller to increase or decrease the forcefulness of the increasing or decreasing pressure within the cavity to better match the changing pressures within the thorax. In various embodiments, the user can provide information to the controller to increase or decrease the maximum pressure during exhale. In various embodiments, the user can provide information to the controller to increase or decrease the low pressure during inhale. In various embodiments, the user can provide information to the controller to increase or decrease the fluctuations in pressure within the cavity. The user can use the interface to provide information to the controller that can help the controller to do a better job of modulating the changing pressure within the cavity to better match the pressure within the thorax. The interface can be used to adjust the performance of the pressure modulator based on the patient's subjective feelings of pain to reduce the patient's feelings of pain.

The manual feedback interface 16 can allows the patient or clinicians to adjust the degree of force applied at each temporal component of the respiratory cycle, or the timing of the counteracting forces. In various embodiments, all major components of the positive and negative time segments can be independently adjustable.

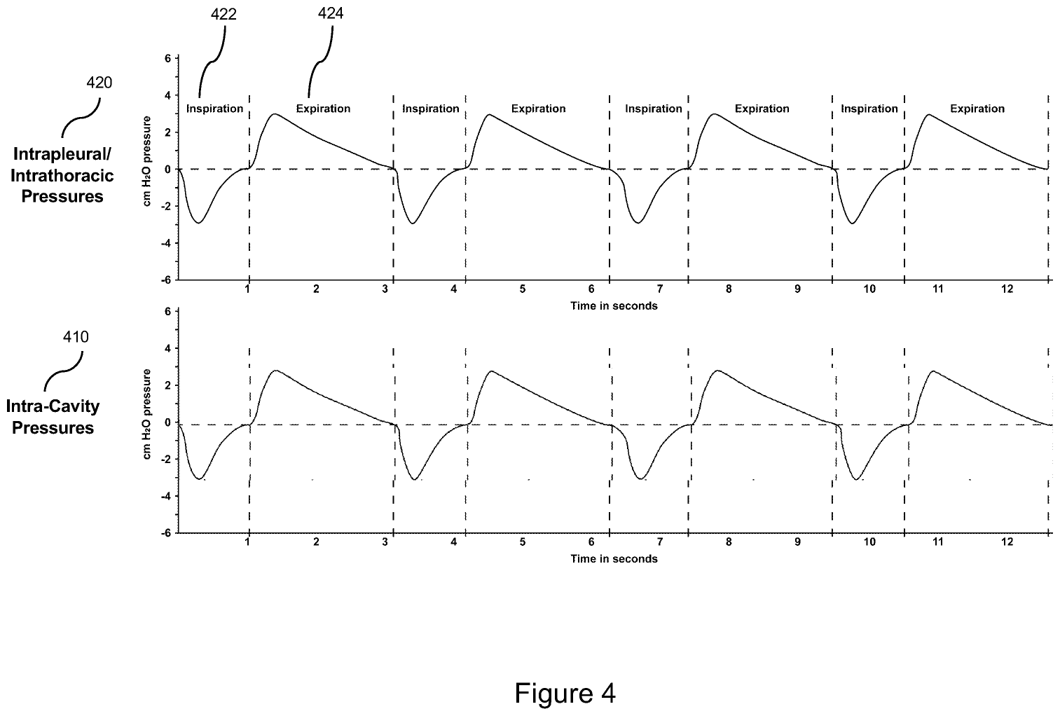

Turning to FIG. 1 and to the ventilatory cycle shown in FIG. 4, in various embodiments, the manual feedback interface 16 can allow the patient and/or clinician to adjust the pressure within the cavity at each portion of the ventilation cycle separately. By way of non-limiting example, the patient and/or clinician can separately adjust the pressure within the cavity at the beginning of inspiration, the middle of inspiration, and/or the end of inspiration. The patent and/or clinician can adjust the pressure within the cavity at various points during inspiration. By way of non-limiting example, the patient and/or clinician can separately adjust the pressure within the cavity at the beginning of exhalation, the middle of exhalation, and the end of exhalation. The patent and/or clinician can adjust the pressure within the cavity at various points during exhalation. By way of non-limiting example, the patient and/or clinician can separately adjust the pressure within the cavity for resting periods after inspiration and before exhalation, and for resting periods after exhalation and before inspiration. The patent and/or clinician can adjust the pressure within the cavity at various points in the respiratory cycle separately.

In various embodiments, the manual feedback interface 16 can be one or more control knobs. By way of non-limiting examples, the manual interface 16 can be a single easy-to-use control knob for adjusting the pressure modulator's response to patient breathing, or can include separate control knobs for adjusting the pressure modulator's response to the patient's inspiration phase and expiration phase. In various embodiments, the manual feedback interface can include similar human-machine-interface technology that allow the patient or provider to adjust the degree of pressure or vacuum at any given segment of the ventilatory cycle. In various embodiments, the manual feedback interface can include a graphical presentation of the current pressure or vacuum pattern to enhance the ability of the patient or provider in these adjustments. The user can use the manual feedback interface 16 to adjust all parts of the positive and negative pressure segments independently, allowing precise control of the pressure within the cavity at all points along the ventilatory cycle. The user can use the manual feedback interface 16 to adjust all parts of the positive and negative pressure time segments independently, allowing precise control of the positive and negative pressure waveforms.

In various embodiments, the controller 10 can receive inputs from the sensor 8, intracavity pressure sensor 18, the paradoxical chest wall motion detector 15, and/or the manual feedback interface 16. The controller can synthesize information from one or more inputs to minimize patient discomfort by modulating the pressure within the cavity throughout the patient breathing cycle.

In various embodiments, the pressure modulator 20 can incorporate cough/sneeze detection with preset response patterns. By way of non-limiting example, the detection mechanism may include an accelerometer that can sense the fast movement of the patient as the patient is sneezing. By detecting a sneeze, the pressure modulator can respond quickly to the rapid increase in pressure caused by a sneeze. A typical sneeze can increase the pressure within the thorax to levels much higher than typically experienced during a breathing cycle, and the sneeze detection system can allow the pressure modulator to quickly respond by increasing the pressure within the cavity to higher levels to match the pressure within the thorax, and then quickly releasing the spike in pressure within the cavity so the pressure modulator can continue to match the pressure within the thorax throughout and after the sneezing event.

Wounds and incisions may have varying shapes. The shape of the device at its attachment to the chest may be customized and/or adjustable so as to be able to overlie the specific wound or incisions. In various embodiments, the rim wall 5 can be adjustable in either length or breadth to allow the pressure modulator to cover a larger or smaller area of the chest wall.

The device may have an internal adjustable strut mechanism capable of applying additional force to the edges of the wound or incision so as to maintain approximation of the edges. The shape can be adjustable as a function of wounds. The pressure modulator can include internal adjustable components that can push on edges of an incision. The internal adjustable components that push on the edges of the incision can vary the force applied to the edges of the incision.

In various embodiments, the device may incorporate battery power from a battery 14 that can be part of a portable pressure modulator and/or the device can receive power from an outside source such as power provided by another piece of equipment or power drawn from a wall socket.

In various embodiments, the rim wall 5 or other components that contact a patient can have a temperature adjustor 17, and the temperature of patient touching components can be adjustable via either warming or cooling capabilities. By way of non-limiting example, in various embodiments, this can include one or more tubes within the rim wall 5 that can allow heated or cooled fluid to be passed through the rim wall to adjust the temperature being felt by the patient.

In various embodiments, specific customized versions of the pressure modulator 20 can be adapted for use after specific surgeries or surgical procedures. By way of non-limiting examples, thoracic and cardiac surgery tends to utilize a specific ensemble of incisions, with the median sternotomy and lateral thoracotomy being the most common. Accordingly, there may be specific versions of the device adapted for specific uses, such as reducing the pain caused by a median sternotomy or lateral thoracotomy. These versions of the pressure modulator can include specific shapes of the pressure modulator, and/or specific shapes of the rim wall so as to be optimized for specific incisions or procedures.

In various embodiments, the pressure modulator may have a harness. In addition to having the location of the pressure modulator stabilized by the patient facing adhesive, the pressure modulator may additionally be stabilized in location by an attached harness. In various embodiments, this harness may incorporate a circumferential belt around the chest and one or more shoulder straps. There may be various designs for this harness, including a design optimized for a precordial location and a design optimized for a sternal location. In addition to assisting in the maintenance of location, the thoracic harness may additionally assist in applying a counter force during phases in which the device intracavity relative pneumatic pressure is greater than atmospheric. At such times, the intracavity pressure may act to push the device off of the patient's chest. The harness can act to provide a counter force to maintain application of the device against the chest.

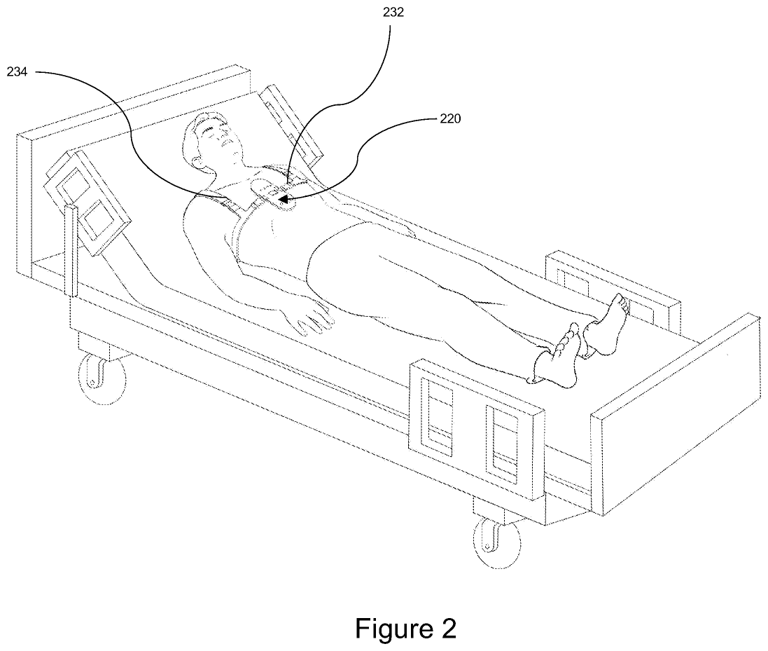

FIG. 2 is a perspective view of a patient laying in a bed with a pressure modulator external to the thorax and applied to minimize pain associated with a median sternotomy incision, according to an illustrative embodiment. The median sternotomy pressure modulator 220 can be a specifically designed pressure modulator adapted for use over a median sternotomy incision. In various embodiments, the median sternotomy pressure modulator can include an elongated and flat shape designed to cover a median sternotomy incision.

At different times in the breathing cycle, the pressure modulator needs to increase the pressure within the cavity to levels above the ambient air pressure and the pressure modulator needs to decrease the pressure within the cavity to levels below the ambient air pressure. In order for the pressure modulator to create pressure levels within the cavity that are different from the ambient air, the pressure modulator must have an airtight seal with the chest wall of the patient. In various embodiments, the pressure modulator 220 can include a circumferential belt 232 and/or a harness 234 to hold the pressure modulator in place against the patient. The pressure modulator can be secured to the patient to form the airtight seal using one or more of an adhesive, a belt 232, or a harness 234.

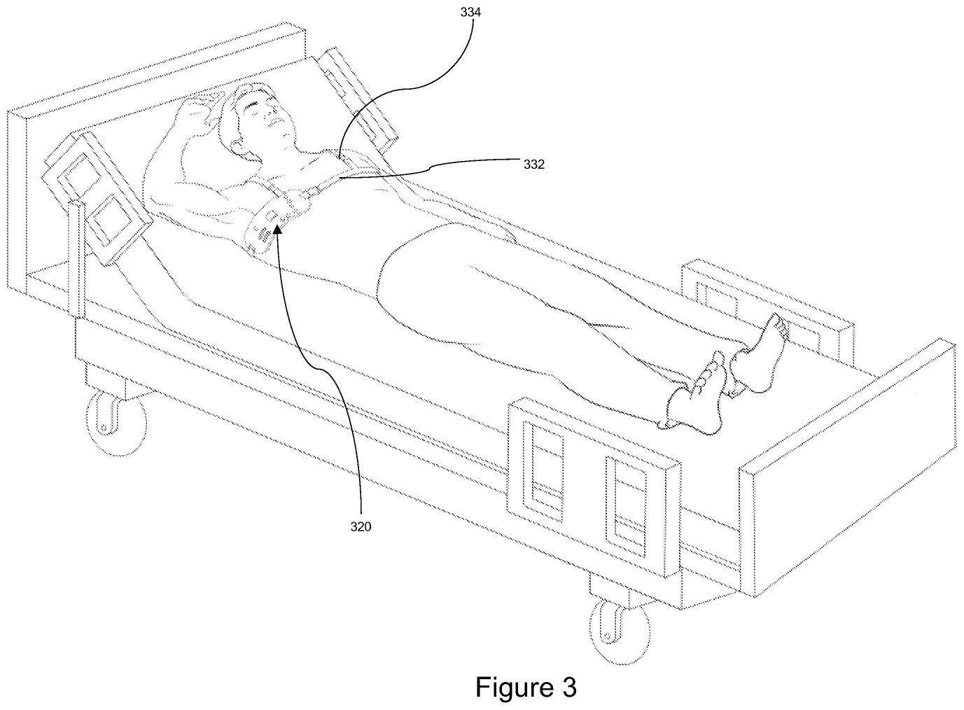

FIG. 3 is a perspective view of a patient laying in a bed with the pressure modulator external to the thorax and applied to minimize pain associated with fractures of the ribs laterally, according to an illustrative embodiment. The lateral rib fracture pressure modulator 320 can be a specifically designed pressure modulator adapted for use over a lateral rib fracture. In various embodiments, the lateral rib fracture pressure modulator can include a curved shape designed to cover the lateral rib area.

In various embodiments, the pressure modulator 320 can include a circumferential belt 232 and/or a harness 234 to hold the pressure modulator in place against the patient. The pressure modulator can be secured to the patient to form the airtight seal using one or more of an adhesive, a belt 232, or a harness 234.

FIG. 4 is chart showing the changes in pressure over time within the thorax and the changes in pressure over time in the cavity of the pressure modulator on the exterior of the thorax, and the relationships between the changing pressures, according to an illustrative embodiment. The pressure within the cavity, as shown in the lower chart 410 is modulated to match the intrathoracic pressure, shown in the upper chart 420. In a normal breathing cycle, the patient breathes in (inspiration phase 422) and the patient breathes out (expiration phase 424) back and forth. In various embodiments, as the patient breathes in, the pressure within the thorax can drop to approximately −3 cm H₂O relative to the ambient air, although various pressures are possible. Different patients may have different maximum or minimum pressures compared to other patients, and the same patient may have different maximum or minimum pressures in different breaths. Faster inspiration can lead to greater negative intrathoracic pressure relative to the ambient air, and slower inspiration can result in reduced negative intrathoracic pressure relative to the ambient air. Using a combination of sensors, flail detectors, and/or manual feedback interface, the controller can direct one or more pumps to modulate the extra-thoracic, device intra-cavity pressures to match, or approximately match, the intrathoracic pressures.

Similarly, as the patient breathes out, the pressure within the thorax can increase to approximately 3 cm H₂O relative to the ambient air, although various pressures are possible. Different patients may have different maximum or minimum pressures compared to other patients, and the same patient may have different maximum or minimum pressures in different breaths. Faster expiration can lead to greater positive intrathoracic pressure relative to the ambient air, and slower expiration can result in reduced positive intrathoracic pressure relative to the ambient air. Using a combination of sensors, flail detectors, and/or manual feedback interface, the controller can direct one or more pumps to modulate the extra-thoracic, device intra-cavity pressures to match, or approximately match, the intrathoracic pressures

As shown in FIG. 4, the intrathoracic pressure fluctuates between positive and negative pressure relative to the ambient air during each breathing cycle, and the pressure modulator can modulate the device intra-cavity pressure in real time to match the changing intrathoracic pressure as the intrathoracic pressure is changing. The device intracavity pressure can be varied in such a manner as to counteract the forces that ventilation apply to the chest wall. The pressure within the cavity can be changed to oppose the pressure changes within the thorax.

In various embodiments that include a manual feedback interface, the manual feedback interface can include a graphical display of the intrathoracic pressure, and a graphical display of the device intra-cavity pressure. In various embodiments, these graphical displays can present the same information, or can resemble, the graphs shown in FIG. 4. The user can use the manual feedback interface 16 to adjust all parts of the positive and negative pressure time segments independently. Put another way, the user can use the manual feedback interface 16 to adjust the pressure at each part of the ventilatory cycle separately. The user can use the manual feedback interface to make adjustments to the pressure modulator's intra-cavity pressure at any temporal position along the intra-cavity pressure graph.

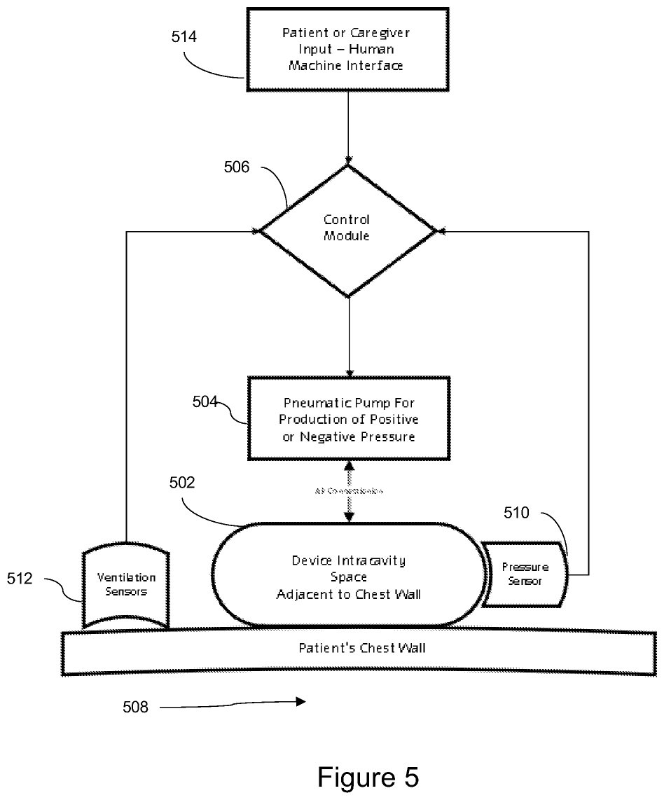

FIG. 5 is a schematic representation of various components of a pressure modulator, according to an illustrative embodiment. The pressure modulator can modulate the pressure within an intracavity space 502 adjacent to a patent's chest wall. A pump 504 can produce positive and negative pressure within the cavity 502. A controller 506 can provide instructions to the pump 504 so that the pump produces positive and negative pressure within the cavity. The controller can provide instructions to the pump to modulate the pressure within the cavity 502 to match the pressure within the patient's intrathoracic space 508.

The control module can receive inputs from sensors that can include an intracavity pressure sensor 510. The intracavity pressure sensor 510 can sense the pressure within the intracavity space 502, and can provide the intracavity pressure information to the controller 506 in real time as the pressure within the cavity is being modulated. The control module can receive inputs from sensors that can include ventilation sensors 512. The ventilation sensors can sense patient ventilation, and can provide the patient ventilation information to the controller in real time as the patient is breathing.

The control module can receive inputs from sensors that can include a manual feedback interface 514. The manual feedback interface 514 can allow a user such as the patient or other user to adjust the pressure modulation. The control module can receive inputs from the manual feedback interface that the control module can incorporate to reduced patient discomfort. The control module can receive inputs from intracavity pressure sensors, ventilation sensors, and/or a manual feedback interface that can allow the control module to direct the pump to modulate the pressure within the intracavity space to minimize patient discomfort. The control module can integrate inputs from various sensors, and can use the inputs that can be integrated together from various sensors to provide an output to the pump to control the pressure within the cavity in a way that provides a dynamic counterforce to patient breathing. The control module can minimize patient discomfort by modulating the intracavity space to match the pressure within the intrathoracic space.

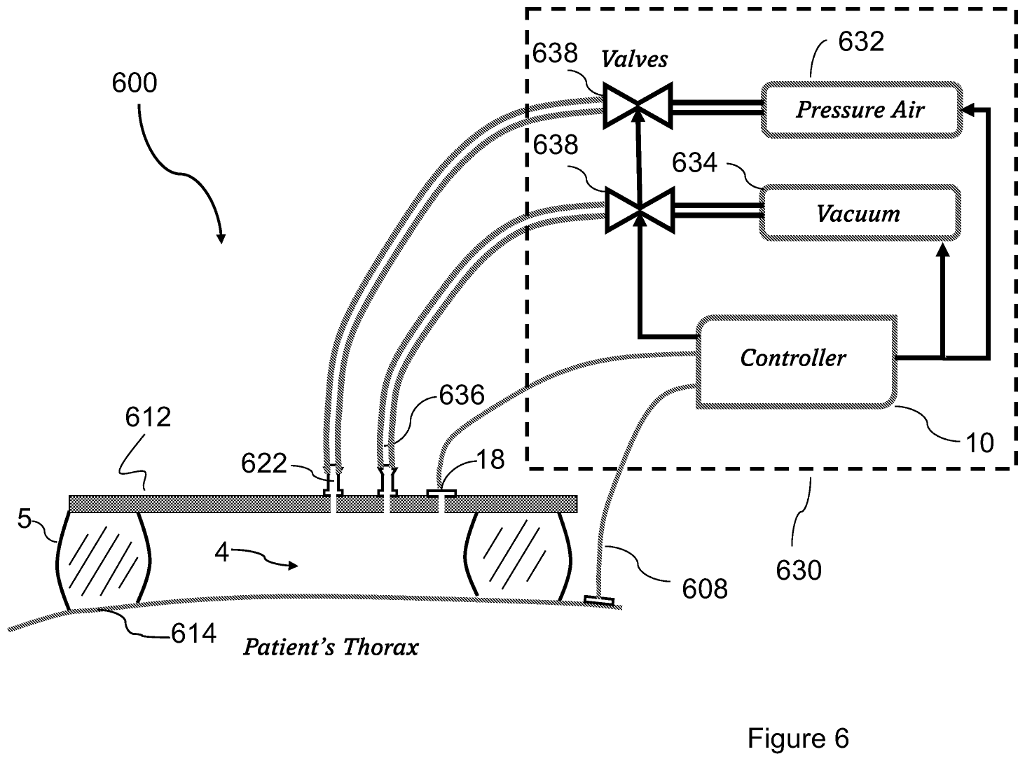

FIG. 6 is a diagrammatic representation of a pressure modulator with a remote base, according to an illustrative embodiment. In various embodiments, various components of a pressure modulator 600 may be housed in a separate remote base 630. Embodiments with a remote base 630 can have a simpler platform 612 that can be lighter weight and easier to apply to a patent.

In embodiments with a remote base, components such as pumps and/or a controller 10 can be located within the remote base. In various embodiments, a pressure modulator may have a single pump that produces increased pressure and reduced pressure (vacuum pressure). In various embodiments, a pressure modulator may have a separate pressure increasing pump 632 and a separate pressure decreasing pump 634. Various hoses 636 can carry compressed air and vacuum to the cavity 4. Various valves 638 can allow the pressure modulator 600 to switch quickly between increased pressure within the cavity 4 that is greater than ambient pressure and reduced pressure within the cavity 4 that is less than ambient pressure. Various valves 638 can be controlled by the controller 10 so that the controller can more effectively modulate the pressure within the cavity 4. Various fittings 622 can allow the platform 612 to be disconnected from and reconnected with the remote base. Various sensors such as a ventilation sensor 608 can be separate from the platform 612, and can provide inputs to the controller wherever the controller is located, including a controller 10 that can be located within the remote base 630.

In various embodiments, the platform 612 can include rim wall 5, an adhesive 614, and/or a harness. The platform 612 can include various components that can help to hold the platform in place and to create an airtight compartment 4. In various embodiments, various other components can be located in a remote base 630.

The pressure modulator can be a system that includes a number of components and capabilities, including:
1. An adjustable stabilizing platform 12 that may be applied to the rib cage 1 of a patient over the chest wall injury, rib fracture 2, flail segment 3 or incision. Other components of the system may be incorporated into this platform. The platform may be adjustable so as to optimize function.
2. The patient-side surface of the pressure modulator can have rim wall 5 that can include a pliable material, a bladder, and/or adhesive at its circumferential edge. Application to the chest wall creates an airtight compartment forming a cavity 4 between the platform itself and the thorax 6.
3. The pressure modulator may have a component 9 such as a pump capable of placing this compartment 4 under negative and positive pressure and of rapidly varying the pressure 11 in the enclosed space so as to minimize the pain or paradoxical motion of chest wall injuries, rib fractures 2, flail segments 3 or incisions. Such a component 9 would be likely to be an air pump and associated valves and connecting hoses.
4. The device may have one or more sensors 8 for measuring ventilation and/or receive inputs 7 from any system that measures patient respiration. Ventilation can be measured using multiple existing technologies, including electrical impedance and plethysmography.
5. A control system 10 capable, in combination with the pump 9, of varying the patient-side compartment pressure 11 in a manner reciprocal to ventilation induced intrathoracic pressure. Thus, this is a system for dynamically opposing the changes in intrathoracic pressure that accompany ventilation. Such a control system 10 can be a standard electrical control system or computer board. The controller may be adjusted by an operator based on the patient's subjective sense of painful paradoxical movement. The controller is capable of synthesizing inputs from the patient and operator so as to minimize paradoxical chest wall motion.
6. A harness system for stabilization during periods of positive pressure within the airtight compartment 4.
7. A battery 14.
8. A patient side sensor 15 capable of detecting or measuring paradoxical chest wall motion.

In various embodiments, the pressure modulator can include:
1. The adjustable stabilizing frame or platform 12.
2. The adjustable circumferential rim wall 5, having a pliable and/or adherent material 13 that is applied to the patient side circumference to create an airtight compartment 4.
3. The pump 9 for varying the pressure 11 within the airtight compartment 4.
4. The component or mechanism 8 for measuring the intrinsic ventilation of the patient.
5. The component or mechanism 8 for receiving inputs 7 from other devices measuring patient ventilation and physiology. Existing technologies such as electrical impedance or plethysmography are readily available for this purpose.
6. The controller 10 that interfaces between the measurement of patient ventilation and the component 9 that varies pressure 11 within the airtight compartment 4.

This disclosure includes a method for treating chest wall injuries, including thoracic incisions, the method comprising: creating a localized airtight compartment external to the chest and fully covering the area of injury; varying the pressure within the compartment real-time so as to provide a dynamic counterforce to the changes in intrathoracic pressure that occur during each ventilatory cycle.

A device can include the real-time sensing of ventilation or chest wall motion and dynamic variation of the pressure within the localized airtight compartment in such a manner that the pressure within the airtight compartment opposes pressure changes within the chest. The device can include real-time sensing of paradoxical flail chest free-segment movement and dynamic variation of the pressure within the localized airtight compartment in such a manner that the pressure within the airtight compartment opposes such paradoxical motion. The device can also incorporate an adherent material on the patient-side surface of the apparatus at its circumference such that an adherent airtight compartment is created between the frame and the thorax. The device can also incorporate an adjustable bladder component on the patient-side surface of the apparatus at its circumference such that the airtight compartment may adjust to chest wall anatomy. The device can also incorporate the capability of receiving input signals from other devices measuring ventilation or chest wall movement. The frame can be adjustable so as to better configure to the shape of the chest wall. The power source can be contained within the device in the form of a battery. The apparatus incorporates a patient side sensor capable of detecting or measuring paradoxical chest wall motion and incorporating that data in the variation of pressure within the airtight compartment. The controller may be adjusted by an operator based on the patient's subjective sense of painful paradoxical movement. The controller can be in the form of general purpose computer and algorithm capable of synthesizing inputs from the patient and operator so as to minimize paradoxical chest wall motion.

This device and method, and the various embodiments are intended to treat injuries to the thorax so as to lessen the pain suffered by the patient or further injury to the chest wall or underlying visceral organs. The pressure modulator disclosed herein provides the capability to noninvasively treat or stabilize chest wall injuries, rib fractures, flail segments or incisions.

The pressure modulator described herein can include the combination of detecting ventilation associated changes in intrathoracic pressure so as to dynamically counteract it via an extrathoracic pneumatic compartment.

Thoracic injuries, such as thoracic surgeries, rib fractures, and flail chest segments, may be treated regionally by means of real-time dynamic alteration of extrathoracic air pressure.

### OTHER PUBLICATIONS INCORPORATED IN THE CURRENT APPLICATION BY REFERENCE

Ciraulo, D. L., et al. “Flail chest as a marker for significant injuries.” J.Am.Coll.Surg. 178 (1994): 466-70.
Dehghan, N., et al. “Flail chest injuries: a review of outcomes and treatment practices from the National Trauma Data Bank.” J.Trauma Acute.Care Surg. 76.2 (2014): 462-68.

### MODIFICATIONS

It will be understood that many changes in the details, materials, steps and arrangements of elements, which have been herein described and illustrated in order to explain the nature of the invention, may be made by those skilled in the art without departing from the scope of the present invention.

Since many modifications, variations and changes in detail can be made to the described preferred embodiment of the invention, it is intended that all matters in the foregoing description be interpreted as illustrative and not in a limiting sense. Thus, the scope of the invention should be determined by the appended claims and their legal equivalents.

1. A pressure modulator for treating pain associated with thoracic incisions and injuries, the pressure modulator comprising:
a platform adapted for creating a localized airtight compartment external to a chest and fully covering an injury area;
a pump configured to create relative positive pressure and relative vacuum within the airtight compartment; and
a controller adapted to cycle intracavity pressure within the localized airtight compartment so that the enclosed space is under relative vacuum pressure relative to ambient air during patient inhalation and relative positive pressure relative to ambient air during patient exhalation.

2. The pressure modulator of claim 1 further comprising a sensor adapted to sense ventilation in real time, wherein the pump dynamically varies the pressure within the localized airtight compartment in response to a sensed ventilation.

3. The pressure modulator of claim 1 further comprising an adherent material on a patient-side surface of the frame, the adherent material adapted to adhere to the chest and form an airtight compartment between the frame and the chest.

4. The pressure modulator of claim 1 further comprising an adjustable bladder on a patient-side surface of the frame at a circumference of the frame such that the airtight compartment is adapted to adjust to chest wall anatomy.

5. The pressure modulator of claim 1 further comprising an intracavity pressure sensor, wherein the controller is adapted to incorporate sensed data from the intracavity pressure sensor to dynamically adjust the intracavity pressure to match changing intrathoracic pressure as the patient is breathing.

6. The pressure modulator of claim 1 further comprising a manual feedback interface adapted to allow an operator to adjust the pressure within the localized airtight compartment based on a patient's subjective sense of pain.

7. The pressure modulator of claim 1 wherein the controller is adapted to integrate inputs from a manual feedback interface and inputs from one or more sensors to control variation of pressure within the airtight compartment, wherein the sensors are adapted to sense ventilation or pressure within the airtight compartment.

8. A pressure modulator for treating chest wall injuries, the device comprising:
a frame adapted for creating a localized airtight compartment external to a chest of a patient and fully covering an area of injury, the frame having a pliable circumferential component with an adhesive;
a sensor;
a controller adapted to control an air pump; and
the air pump, wherein the controller is capable of dynamically varying the pressure within the compartment in real-time in response to data from the sensor so as to provide a dynamic counterforce to the changes in intrathoracic pressure that occur during each ventilatory cycle.

9. The pressure modulator of claim 8 wherein the sensor is a sensor for detecting ventilation or chest wall motion and, the air pump capable of dynamic variation of the pressure within the localized airtight compartment varies the pressure within the airtight compartment to oppose pressure changes within the chest.

10. The pressure modulator of claim 8 wherein the sensor is a sensor for detecting chest movement, and the air pump capable of dynamic variation of the pressure within the localized airtight compartment varies the pressure within the airtight compartment in response to such motion.

11. The pressure modulator of claim 8 further incorporating an adjustable bladder component on the patient-side surface of the apparatus at its circumference such that the airtight compartment may adjust to chest wall anatomy.

12. The pressure modulator of claim 8 further comprising an input receiver capable of receiving input signals from other devices measuring ventilation or chest wall movement.

13. The pressure modulator of claim 8 wherein the frame is adjustable to conform to the shape of the chest wall.

14. The pressure modulator of claim 8 further comprising a patient side sensor capable of detecting or measuring chest wall motion and incorporating that data in the variation of pressure within the airtight compartment.

15. The pressure modulator of claim 8 further comprising a manual feedback interface for adjusting the variation of pressure within the airtight compartment by an operator based on the patient's subjective sense of pain.

16. The pressure modulator of claim 8 further comprising a controller adapted to integrate inputs from the sensor and inputs from the operator so as to minimize the patient's subjective sense of pain.

17. A pressure modulator for treating chest wall injuries, the device comprising:
a localized airtight compartment adapted to be placed external to a chest and fully covering an area of injury;
a pump adapted to vary a pressure within the localized airtight compartment in real-time so as to provide a dynamic counterforce to changes in intrathoracic pressure that occur during each ventilatory cycle, the dynamic counterforce minimizing a patient's sense of pain by providing a pressure in the localized airtight compartment that opposes movement of the chest wall injury caused by patient breathing.

18. The pressure modulator of claim 17, further comprising a sensor adapted to sense in real time ventilation or chest wall motion, wherein the pump is adapted to dynamically vary the pressure within the localized airtight compartment in response to sensed ventilation or chest wall motion data in such a manner that the pressure within the airtight compartment opposes pressure changes within the chest.

19. The pressure modulator of claim 17, further comprising a sensor adapted to sense in real time patient ventilation, wherein the pump is adapted to dynamically vary the pressure within the localized airtight compartment in response to sensed patient ventilation data in such a manner that the pressure within the airtight compartment opposes intrathoracic pressure.

20. The pressure modulator of claim 17, further comprising a manual feedback interface adapted to be adjusted by a user based on a patient's subjective sense of pain, wherein the pump is adapted to dynamically vary the pressure within the localized airtight compartment in response to data from the manual feedback interface in such a manner that the pressure within the airtight compartment opposes intrathoracic pressure.
